(19) United States
(12) Patent Application Publication
(10) Pub. No.: US 2025/0334392 A1
(43) Pub. Date: Oct. 30, 2025

(54) AN INTERFEROMETER FOR TEAR FILM MEASUREMENT WITH SUB-MICRON RESOLUTION

(71) Applicant: THE UAB RESEARCH FOUNDATION, BIRMINGHAM, AL (US)

(72) Inventors: Yuqiang Bai, Vestavia, AL (US); Jason Nichols, Birmingham, AL (US)

(21) Appl. No.: 19/189,736

(22) Filed: Apr. 25, 2025

Related U.S. Application Data
(60) Provisional application No. 63/638,526, filed on Apr. 25, 2024.

(51) Int. Cl. G01B 9/02; A61B 3/10; G01B 9/02001; G01B 9/02015; G01B 9/02017; G01B 9/02055

(52) U.S. Cl. CPC G01B 9/02083; A61B 3/101; G01B 9/02001; G01B 9/02019; G01B 9/02028; G01B 9/0203; G01B 9/02076

(57) ABSTRACT

Disclosed are various systems and methods of using interferometry to measure the tear film for disease prediction and treatment. A point-by-point scan of a corneal surface of an eye is obtained from an interferometry system, including at least an interference signal. Next, a large field-of-view of the corneal surface is obtained from an objective lens with a curved focal plane matched to a curvature of the corneal surface. Subsequently, motion correction is performed on the point-by-point scan. Then, noise is filtered from the interference signal. A tear film lipid layer signal and a precorneal tear film signal are separated from the filtered interference signal. Later, a best fit frequency for the tear film lipid layer signal and the precorneal tear film signal are determined. Then, a thickness of the tear film lipid layer and the precorneal tear film are determined based at least in part on the best fit frequency.

### CROSS-REFERENCE TO RELATED APPLICATIONS

This application claims the benefit of and priority to U.S. Provisional Application Ser. No. 63/638,526, filed Apr. 25, 2024, entitled “INTERFEROMETER FOR TEAR FILM MEASUREMENT WITH SUB-MICRON RESOLUTION,” which is incorporated herein by reference in its entirety.

### STATEMENT REGARDING FEDERALLY SPONSORED RESEARCH OR DEVELOPMENT

This invention was made with government support under grant number EY033029 awarded by National Institutes of Health. The government has certain rights in this invention.

### BACKGROUND

In humans, the precorneal tear film (PCTF) is a thin layer (˜3-5 μm) of a complex biological fluid coating the ocular surface, that serves to nourish and protect the ocular surface and provide a smooth refractive optical surface for vision. In dry eye disease, the PCTF becomes thinner, and destabilizes (evaporates) rapidly leading to hyperosmolarity, inflammation, and ocular surface desiccation. More than 30 million people in the United States are impacted by dry eye disease and the economic burden to society is estimated to be over $50 billion. Dry eye disease continues to be a challenge to diagnose, monitor, and treat because many dry eye tests are conducted inconsistently, lack sufficient reliability or accuracy, and do not correlate with symptoms of the disease. Clinical measures of tear film dynamics (thinning and breakup) are subjective in nature and generally lack validity and repeatability.

### SUMMARY

In accordance with the purpose(s) of this disclosure, as embodied and broadly described herein, the disclosure, in various aspects, relates to an interferometer for tear film measurement with sub-micron resolution and methods of use thereof. The solutions described herein combine various mechanical, biological, and ophthalmological principles to arrive at novel solutions for measuring the tear film of the human eye.

Aspects of the present disclosure provide for systems and methods of using interferometry to measure the tear film for disease prediction and treatment. Embodiments of the present disclosure include: a system having a computing device with a processor and a memory, and machine-readable instructions stored in the memory which, when executed by the processor, cause the computing device to perform multiple steps to control an interferometry system and to process the signals received from the interferometry system. For example, the machine-readable instructions can cause the computing device to at least obtain a point-by-point scan of a cornea of an eye from an interferometry system, where the point-by-point scan includes at least an interference signal. In addition, the computing device can perform motion correction on the point-by-point scan and filter noise from the interference signal. In some examples, the computing device can separate a tear film lipid layer signal and a precorneal tear film signal from the filtered interference signal, determine a best fit frequency for the tear film lipid layer signal and the precorneal tear film signal, and determine a thickness of the tear film lipid layer and a thickness of the precorneal tear film based at least in part on the best fit frequency. The computing device can perform motion correction by recording motion of the cornea during the point-by-point scan to produce a motion record, identifying one or more features of the cornea from the motion record, comparing a respective position of the one or more features of the cornea in the motion record to the point-by-point scan, and correcting the respective position of the one or more features in the point-by-point scan based at least in part on a detected change in the respective position between the motion record and the point-by-point scan. The computing device can perform noise filtering by subtracting the interference signal from a standard interference signal.

According to various examples, the interferometry system can include a laser configured to produce a laser beam; an axicon-pair configured to produce a hollow beam from the laser beam; a scanning mirror to direct the hollow beam toward an imaging lens; a beam splitter configured to receive a reflected beam from the cornea of the eye and split the reflected beam; and a line scan camera to receive the split beam. The interferometry system can further include an area camera configured to record motion of the cornea. In some examples, the imaging lens is an objective lens which produces a curved focal plane similar to the surface of a cornea.

A method is provided which can comprise the steps of obtaining, with an interferometry system, a point-by-point scan of a cornea of an eye, the point-by-point scan including at least an interference signal, performing motion correction on the point-by-point scan; filtering noise from the interference signal; separating a tear film lipid layer signal and a precorneal tear film signal from the filtered interference signal; determining a best fit frequency for the tear film lipid layer signal and the precorneal tear film signal; and determining a thickness of the tear film lipid layer and a thickness of the precorneal tear film based at least in part on the best fit frequency. In some examples, performing motion correction on the point-by-point scan further includes recording motion of the cornea during the point-by-point scan to produce a motion record; identifying one or more features of the cornea from the motion record; comparing a respective position of the one or more features of the cornea in the motion record to the point-by-point scan; and correcting the respective position of the one or more features in the point-by-point scan based at least in part on a detected change in the respective position between the motion record and the point-by-point scan. Recording motion of the cornea can use an area camera. In some examples, filtering noise from the interference signal further includes at least subtracting the interference signal from a standard interference signal. According to various examples, obtaining, with the interferometry system, the point-by-point scan can include the steps of producing a laser beam with a laser; producing a hollow beam from the laser beam using an axicon-pair; directing the hollow beam toward an imaging lens using a scanning mirror; receiving a reflected beam from the cornea of the eye with a beam splitter; splitting the reflected beam with the beam splitter to produce a split beam; and receiving the split beam with a line scan camera. The imaging lens can have a curved focal plane similar to the surface of a cornea.

Further, a system is provided having at least an interferometry system, a computing device, comprising a process and a memory, and machine-readable instructions stored in the memory which, when executed by the processor, cause the computing device to perform a number of steps. The computing device can obtain an interference signal from a cornea of an eye using the interferometry system and filter noise from the interference signal to produce a deduction signal. In addition, the computing device can separate a tear film lipid layer signal and a precorneal tear film signal from the deduction signal. According to some examples, the computing device can determine a best fit frequency for both of the tear film lipid layer signal and the precorneal tear film signal and determine a thickness of the tear film lipid layer and a thickness of the precorneal tear film based at least in part on the respective best fit frequencies. The computing device can further use an area camera of the interferometry system to record motion of the cornea to generate a motion record and perform motion correction on the interference signal based at least in part on the motion record. In some examples, when performing motion correction, the computing device can identify one or more features of the cornea from the motion record, compare a respective position of the one or more features of the cornea in the motion record to the interference signal; and correct the respective position of the one or more features in the interference signal based at least in part on a detected change in the respective position between the motion record and the point-by-point scan. When obtaining an interference signal from a cornea of an eye using the interferometry system, the computing device can direct a hollow laser beam of the interferometry system toward the cornea through an imaging lens and receive a reflected beam from the cornea of the eye to obtain the interference signal. The imaging lens can be an objective lens having a curved focal plane similar to the surface of a cornea. When filtering noise from the interference signal, the computing device can subtract the interference signal from a standard interference signal. Additionally, when the computing device determines a best fit frequency for both of the tear film lipid layer signal and the precorneal tear film signal, the computing device can simulate a plurality of frequency curves and perform curve-fitting with the plurality of frequency curves to determine a best fit frequency.

Other systems, methods, devices, features, and advantages of the devices and methods will be or become apparent to one with skill in the art upon examination of the following drawings and detailed description. It is intended that all such additional systems, methods, devices, features, and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a schematic layout of the interferometric system according to various embodiments of the present disclosure. The light path in the laser point-scanning system is shown, along with the auxiliary imaging system. The two light sources are separated with different polarization states.

FIGS. 2A-D show a schematic of motion correction according to various embodiments of the present disclosure. FIG. 2A is a representative lipid layer image. FIGS. 2B-2C show the schematic movement of the lipid layer images between two adjacent B-scans. FIG. 2D shows displacement calculation from the center of the images.

FIG. 3 shows a prototype diagram of the customized objective, with a field of view of 7 mm² and collinearity of the lens chief rays to the 7.8 mm sphere radiuses, according to various embodiments of the present disclosure.

FIGS. 4A-C show data processing from a precorneal tear film in vivo, according to various embodiments of the present disclosure. FIG. 4A shows recorded spectra from the eye, R_{eye}, and from the reflectance standard, R_{std}. FIG. 4B shows the interference oscillation was derived from the difference between the spectra from the eye and from the reflectance standard. FIG. 4C shows the derived spectral profile was decomposed into two components. The curve-fitting analysis was carried out to retrieve the thickness of the lipid layer (LL) and precorneal tear film (PCTF) individually. The original spectra are shown by solid line, and the simulated curves are shown by the broken line. The plots of LL are vertically shifted.

FIG. 5 is a flowchart illustrating one example of functionality implemented as a portion of an application executed in the interferometric system of FIG. 1 according to various embodiments of the present disclosure.

FIG. 6 is a is a flowchart illustrating one example of functionality implemented as a portion of an application executed in the interferometric system of FIG. 1 according to various embodiments of the present disclosure.

FIG. 7 is a flowchart illustrating one example of functionality implemented as a portion of an application executed in the interferometric system of FIG. 1 according to various embodiments of the present disclosure.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, interferometry, ophthalmological, computer processing techniques and the like, which are within the skill of the art. Such techniques are explained fully in the literature.

The following examples are put forth to provide those of ordinary skill in the art with a complete disclosure and description of how to perform the methods and use the compositions and compounds disclosed and claimed herein. Efforts have been made to ensure accuracy with respect to numbers (e.g., amounts, measurements, etc.), but some errors and deviations should be accounted for.

Before the embodiments of the present disclosure are described in detail, it is to be understood that, unless otherwise indicated, the present disclosure is not limited to particular materials, machines, computing processes, or the like, as such can vary. It is also to be understood that the terminology used herein is for purposes of describing particular embodiments only and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

It should be noted that ratios, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

### Discussion

Disclosed are various approaches for an interferometer for tear film measurement with sub-micron resolution. Quantifying the thickness dynamics of the human tear film facilitates understanding of its structural and functional roles in ocular health and ocular surface disease. Disclosed is a powerful new imaging system to better assess the dynamics of the human precorneal tear film (P CTF). The PCTF is a thin layer of a complex biological fluid coating the corneal surface, destabilization of which can cause dry eye disease (DED). Dry eye disease (DED) is a common ocular condition of the tears and ocular surface associated with eye discomfort, visual disturbance, and painful recurrent corneal erosion and infections that can lead to visual loss in its most severe forms. Today, DED affects more than 30 million people in the United States, with the related healthcare and economic burden estimated to be more than $50 billion. Despite its widespread and increasing prevalence, effects on vision-related quality of life, and potential for sight-threatening complications, the diagnosis, monitoring, and treatment of DED continues to present a significant challenge. Studies have revealed better quantification of PCTF dynamics as a priority to understand the progression of DED and more accurately diagnose and monitor the disease. Evaporation has been identified as the primary mechanism of PCTF decay, which increases tear film osmolarity. Further, a non-uniform distribution of the PCTF and its related decay on the ocular surface have been indicated as well. Findings from these studies collectively helped confirm a key triggering mechanism in DED: recurrent, rapid decay of PCTF occurs on certain areas of the ocular surface (e.g., where tear film breakup or dry spots appear), causing frequent local spikes of osmolarity that may ultimately lead to desiccation and inflammation-related damage of these regions. However, current clinical tests of PCTF dynamics, including the tear film breakup time test, infrared thermography, and wavefront aberrometry, lack sufficient resolution and regional sensitivity to detect discrete changes across the PCTF.

Accordingly, various embodiments of the present disclosure are directed to systems and methods for using interferometry to measure the thickness of the tear film. This novel method utilizes a broadband approach to achieve unprecedented resolution while avoiding issues related to intensity and phase noise of the light source.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

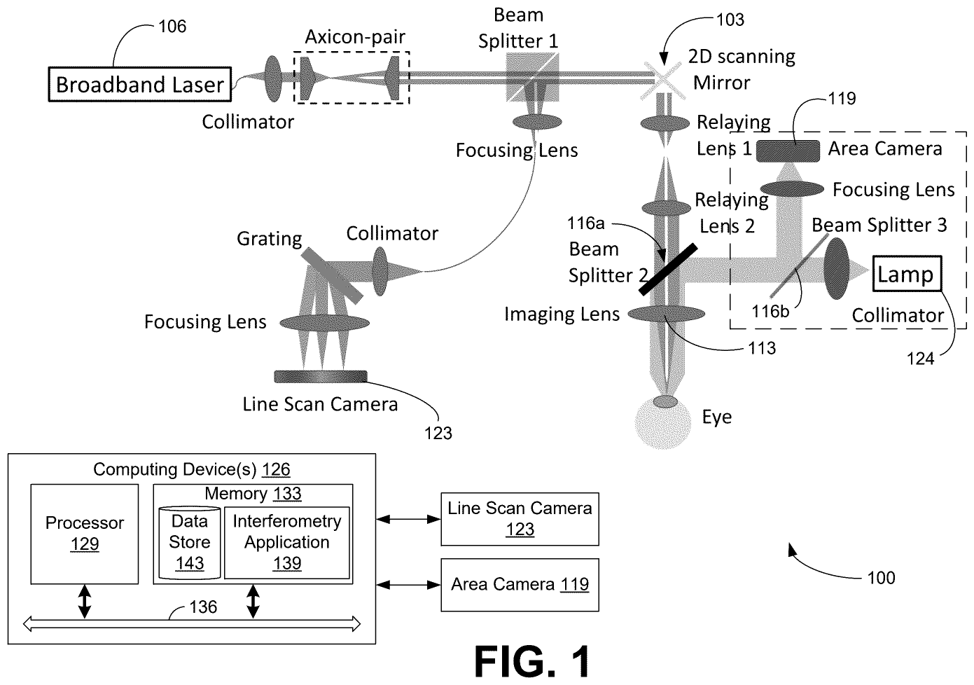

With reference to FIG. 1, the system includes a point-scanning interferometry system 100 which uses a pair of galvanometer scanning mirrors 103 and a supercontinuum (SC) light source 106. The light source 106 can comprise an SC light source which possesses a bandwidth that is as broad as a white-light lamp but with the collimating and focusing properties of a laser. In some embodiments, the light source 106 is a broadband laser. The interferometry system 100 can further include an axicon-pair 109 which can convert the beam from the light source 106 into a hollow beam. The hollow beam can be directed to one or more scanning mirrors 103. In some examples, the scanning mirrors 103 can represent a pair of galvanometer scanning mirrors 103. The scanning mirrors 103 can be rotatable and configured to direct the hollow beam through one or more lenses. In some embodiments, the interferometry system 100 can include an imaging lens 113 which has a curved focal field to image the entire corneal surface. In some examples, the imaging lens 113 is an objective lens which has a curved focal plane similar to the surface of the cornea. The imaging lens 113 can have a focal plane, the curvature of which is matched to that of a cornea surface, thereby producing a focal plane similar to the surface of the cornea. By causing the scanning mirrors 103 to rotate, the hollow beam can be directed through imaging lens 113 to scan each point on the cornea at an angle perpendicular to the surface of the cornea. Additionally, the proposed system can include a customized objective lens as the imaging lens 113, permitting a wide field-of-view of the PCTF over the ocular surface.

In some examples, the interferometry system 100 includes a beam splitter 116a which can direct part of the reflected beam back through the scanning mirrors 103 and into a line scan camera 123. The line scan camera 123 can use the part of the reflected beam to generate the point-by-point scan. In some examples, the line scan camera 123 can use the part of the reflected beam to generate an interference signal. In some examples, the interferometry system 100 includes a lamp 124 which outputs the beam onto the surface of the cornea, and another beam splitter 116b which receives the reflected beam from the surface of the cornea and diverts part of the reflected beam into an area camera 119. The area camera 119 can use the part of the reflected beam to generate a motion record. In some examples, the area camera 119 can record the motion of the cornea during the point-by-point scan conducted by the interferometry system 100.

By using such a system, it is now possible to quantify sub-micron changes in the PCTF and map its spatial distribution across the ocular surface. This has potential to substantially improve the diagnosis, assessment, and treatment of DED, in addition to aiding in the understanding of the underlying mechanisms of the disease.

The system can further include a computing device 126 having a processor 129 and a memory 133. The computing device 126 includes at least one processor circuit, for example, having a processor 129 and a memory 133, both of which can be coupled to a local interface 136. To this end, each computing device 126 may include, for example, at least one server computer or like device. The local interface 136 may include, for example, a data bus with an accompanying address/control bus or other bus structure as can be appreciated.

Stored in the memory 133 are both data and several components that are executable by the processor 129. In particular, stored in the memory 133 and executable by the processor 129 are an interferometry application 139, and potentially other applications. Also stored in the memory 133 can be a data store 143 and other data. In addition, an operating system can be stored in the memory 133 and executable by the processor 129.

The interferometry application 139 includes a number of machine-readable instructions that cause the computing device 126 to perform various functions. For example, the interferometry application 139 can maintain data communication with the line scan camera 123 and the area camera 119. This can allow the interferometry application 139 to obtain scan data, such as a point-by-point scan and an interference signal, from the line scan camera 123 as well as a motion record from the area camera 119. In addition, the interferometry application 139 can send commands to the line scan camera 123, the area camera 119, or other components of the interferometry system 100 to capture readings, perform certain actions, and other commands.

In addition, the interferometry application 139 can obtain an interference signal from a corneal surface of an eye using the interferometry system 100. Then, the interferometry application 139 can filter noise from the interference signal to produce a deduction signal, by isolating the signal from the noise. When filtering noise from the interference signal, the interferometry application 139 can subtract the interference signal from a standard interference signal. In addition, the interferometry application 139 can separate a tear film lipid layer signal and a precorneal tear film signal from the deduction signal. According to some examples, the interferometry application 139 can determine a best fit frequency for both of the tear film lipid layer signal and the precorneal tear film signal and determine a thickness of the tear film lipid layer and a thickness of the precorneal tear film based at least in part on the respective best fit frequencies. The interferometry application 139 can determine the best fit frequency by simulating a plurality of frequency curves and perform curve-fitting with the plurality of frequency curves to determine a best fit frequency.

The interferometry application 139 can further use an area camera 119 of the interferometry system 100 to record motion of the cornea to generate a motion record and perform motion correction on the interference signal based at least in part on the motion record. In some examples, when performing motion correction, the interferometry application 139 can identify one or more features of the cornea from the motion record, compare a respective position of the one or more features of the cornea in the motion record to the interference signal, and correct the respective position of the one or more features in the interference signal based at least in part on a detected change in the respective position between the motion record and the point-by-point scan. When obtaining an interference signal from a cornea of an eye using the interferometry system 100, the interferometry application 139 can direct a hollow laser beam of the interferometry system 100 toward the cornea through an imaging lens 113 and receive a reflected beam from the cornea of the eye to obtain the interference signal.

It is understood that there may be other applications that are stored in the memory 133 and are executable by the processor 129 as can be appreciated. Where any component discussed herein is implemented in the form of software, any one of a number of programming languages may be employed such as, for example, C, C++, C #, Objective C, Java®, JavaScript®, Perl, PHP, Visual Basic®, Python®, Ruby, Flash®, or other programming languages.

A number of software components are stored in the memory 133 and are executable by the processor 129. In this respect, the term “executable” means a program file that is in a form that can ultimately be run by the processor 129. Examples of executable programs may be, for example, a compiled program that can be translated into machine code in a format that can be loaded into a random access portion of the memory 133 and run by the processor 129, source code that may be expressed in proper format such as object code that is capable of being loaded into a random access portion of the memory 133 and executed by the processor 129, or source code that may be interpreted by another executable program to generate instructions in a random access portion of the memory 133 to be executed by the processor 129, etc. An executable program may be stored in any portion or component of the memory 133 including, for example, random access memory (RAM), read-only memory (ROM), hard drive, solid-state drive, Universal Serial Bus (USB) flash drive, memory card, optical disc such as compact disc (CD) or digital versatile disc (DVD), floppy disk, magnetic tape, or other memory components.

The memory 133 is defined herein as including both volatile and nonvolatile memory and data storage components. Volatile components are those that do not retain data values upon loss of power. Nonvolatile components are those that retain data upon a loss of power. Thus, the memory 133 may include, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, or other memory components, or a combination of any two or more of these memory components. In addition, the RAM may include, for example, static random access memory (SRAM), dynamic random access memory (DRAM), or magnetic random access memory (MRAM) and other such devices. The ROM may include, for example, a programmable read-only memory (PROM), an erasable programmable read-only memory (EPROM), an electrically erasable programmable read-only memory (EEPROM), or other like memory device.

Also, the processor 129 may represent multiple processors 129 or multiple processor cores and the memory 133 may represent multiple memories 133 that operate in parallel processing circuits, respectively. In such a case, the local interface 136 may be an appropriate network that facilitates communication between any two of the multiple processors 129, between any processor 129 and any of the memories 133, or between any two of the memories 133. The local interface 136 may include additional systems designed to coordinate this communication, including, for example, performing load balancing. The processor 129 may be of electrical or of some other available construction.

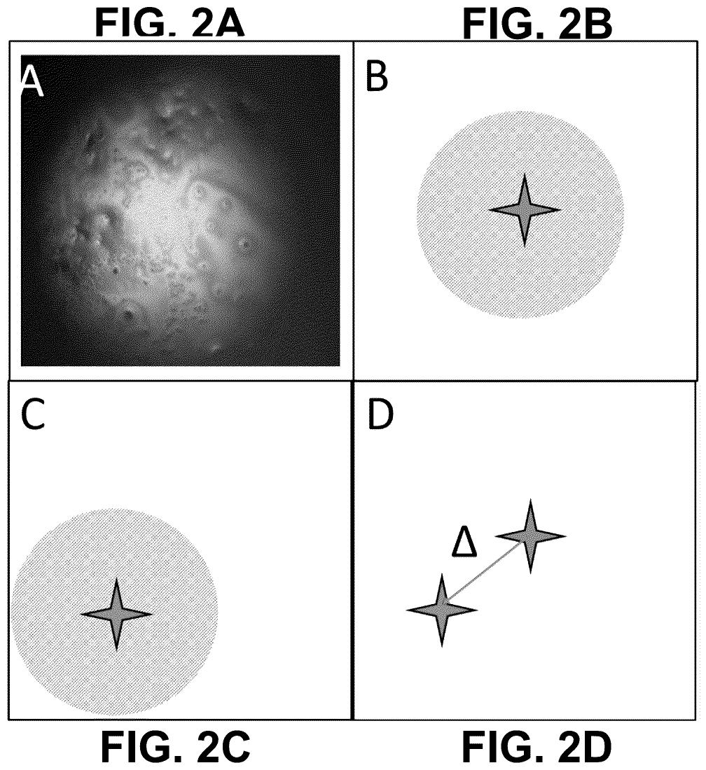

Moving now to FIGS. 2A-D, shown is an example of the motion correction feature of the interferometry application 139. In some examples, when performing motion correction, the interferometry application 139 can identify one or more features of the cornea from the motion record, compare a respective position of the one or more features of the cornea in the motion record to the interference signal, and correct the respective position of the one or more features in the interference signal based at least in part on a detected change in the respective position between the motion record and the point-by-point scan. FIG. 2A shows a representative lipid layer image which can be used to identify one or more features. FIGS. 2B-C show the schematic movement of the lipid layer images between two adjacent B-scans. FIG. 2D shows the displacement calculation from the center of the images which can be used to correct the respective features in the interference signal. A post-processing algorithm can be incorporated into the interferometry application 139 similar to that used to correct motion artifacts in OCT retinal images. During OCT imaging, a scanning laser ophthalmoscopy (SLO) scan is performed with each OCT B-scan. Transverse eye motion is extracted from the serial SLO images, and a motion adjustment vector is assigned to each OCT A-scan. In the system proposed here, the lipid layer imaging system, or area camera 119, will substitute SLO. The proposed interferometry system 100 will be pre-coregistered with the auxiliary lipid layer system. During measurement, the lipid layer image will be acquired simultaneously with each B-scan. The center of the lipid layer image will be retrieved after defining the image boundary. This parameter will facilitate calculation of the displacement among each B-scan by comparing the center position of the adjacent lipid layer images (e.g., the images acquired at current and the next B-scan). Finally, the calculated displacement will be evenly divided by the intensity of B-scan (e.g., 512 points/line), and the divided displacement assigned to each A-scan. In some examples, the power of the illumination light will be set as 2.80 mW, 20 times lower than the calculated maximum power limit.

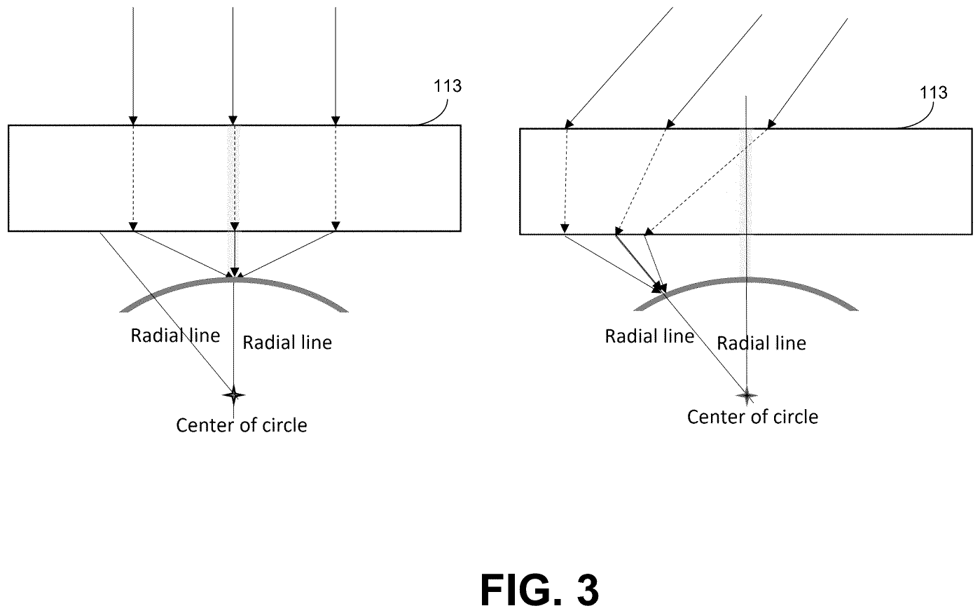

Next, at FIG. 3, shown is a prototype diagram of the customized objective imaging lens 113 with a field of view of 7 mm² and collinearity of the lens chief rays to the 7.8 mm sphere radiuses. The objective lens described herein was designed to image the human corneal surface of approximately 7×7 mm. In some examples, the imaging lens 113 has a curved focal plane with a curvature of 7.8 mm in radius, which is matched to the base radius of curvature of the human cornea.

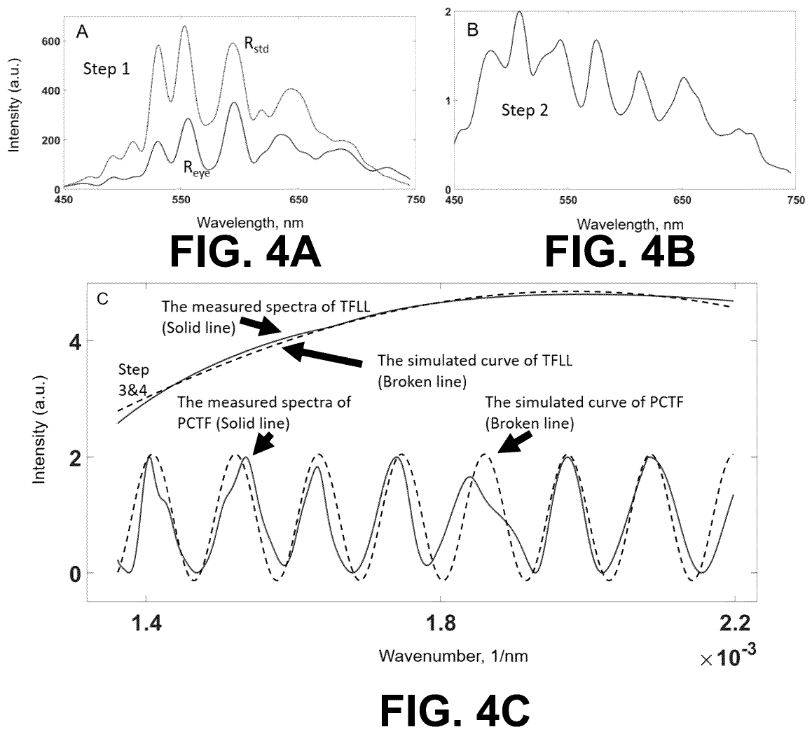

Moving now to FIGS. 4A-C, shown is an example of data processing by the interferometry application 139. FIG. 4A shows recorded spectra from the eye, R_{eye}, and from the reflectance standard, R_{std}. FIG. 4B shows the interference oscillation was derived from the difference between the spectra from the eye and from the reflectance standard. FIG. 4C shows the derived spectral profile was decomposed into two components. The curve-fitting analysis was carried out to retrieve the thickness of the lipid layer (LL) and precorneal tear film (PCTF) individually. The original spectra are shown by solid line, and the simulated curves are shown by the broken line. The plots of LL are vertically shifted.

By isolating the signal of PCTF from the noise, the interferometry application 139 can result in superior accuracy. With this algorithm, PCTF thicknesses as low as ˜100 nm on the central cornea can be readily detected via a customized fiber-based interferometer. This system can use a broad bandwidth approach to achieve unprecedented resolution (˜0.33 μm), which is not possible with other techniques, such as optical coherence tomography (OCT). A broadband source in OCT usually causes dispersion mismatch between the reference and signal arm, a problem difficult to resolve. The bandwidth of the supercontinuum (SC) source used in OCT is tailored to balance the resolution and dispersion problems. However, the present method avoids the dispersion mismatch with direct detection of the interference signal caused by the ocular surface itself, distinct from the reference arm or common path of OCT.

The new interferometry application 139 is used to take full advantage of light bandwidth. A fast Fourier transform (FFT) algorithm causes multiple sub-peaks (side-lobes) with a non-Gaussian spectrum. These side-lobes deleteriously affect the accuracy and sensitivity of OCT, even when a broad source is applied. However, the interferometry application 139 isolates PCTF information from the reflected signal and analyzes it using a curve-fitting method, resulting in ultra-high resolution and superior accuracy. Additionally, ultra-high resolution techniques, such as polarization-sensitive OCT and spectral domain phase microscopy, require outstanding performance on phase stability, posing a challenge to current light sources. The proposed method better tolerates the phase fluctuations of the light source because it only requires limited interferometric stability within an ultra-thin film of ˜3 μm. In other words, any light source with temporal coherence length over a double-pass of 3 μm meets the requirements of this system.

The detected signal (R_{eye}) can be subtracted from the light source's spectral profile (Rₛₜₐ) and modeled as a superposition of two sinusoidal functions. After decomposing the superposition model, a curve-fitting algorithm can retrieve the lipid layer (LL, the outmost layer of tear film) and PCTF thicknesses from the separated functions. Briefly, a simulated plot, Acos(4πnn_{X}T+B) exp (CC_{X})+D, can be applied to fit the separated profile, where nn represents the refractive index of tear film, x represents the wavenumber, and B represents the thickness of tear film. Parameters A, B, C, and _{DD} are varied to determine the best match.

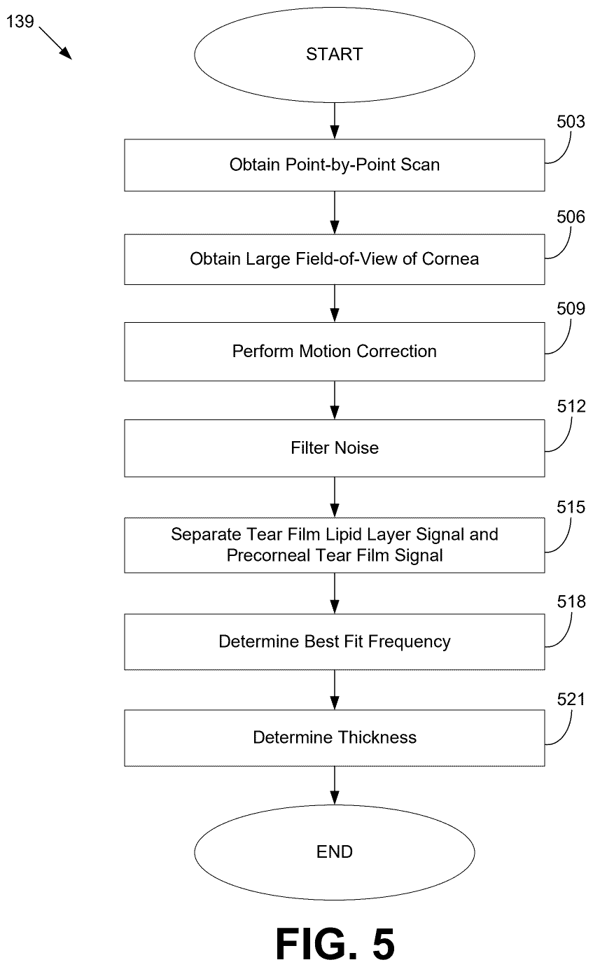

Referring now to FIG. 5, shown is a flowchart that provides one example of the operation of a portion of the interferometry application 139. The flowchart of FIG. 5 provides merely an example of the many different types of functional arrangements that can be employed to implement the operation of the depicted portion of the interferometry application 139. As an alternative, the flowchart of FIG. 5 can be viewed as depicting an example of elements of a method implemented within the interferometry system 100.

Beginning with block 503, the interferometry application 139 can be executed to obtain a point-by-point scan of a corneal surface of an eye. In some examples, the point-by-point scan can include at least an interference signal.

At block 506, the interferometry application 139 could then be executed to obtain a large field-of-view of a cornea of an eye from an objective lens with a curved focal plane matched to a curvature of a corneal surface. In some examples, the objective lens has a curved focal plane of the radius of 7.8 mm.

At block 509, the interferometry application 139 could be executed to perform motion correction on the point-by-point scan.

At block 512, the interferometry application 139 could be executed to filter noise from the interference signal. In some examples, the interferometry application 139 can be executed to filter noise from the interference signal to produce a deduction signal by isolating the signal from the noise. In some examples, the interferometry application 139 can be executed to subtract the interference signal from a standard interference signal.

At block 515, the interferometry application 139 could then be executed to separate a tear film lipid layer signal and a precorneal tear film signal from the filtered interference signal.

At block 518, the interferometry application 139 could then be executed to determine a best fit frequency for the tear film lipid layer signal and the precorneal tear film signal. In some examples, the interferometry application 139 can be executed to determine the best fit frequency by simulating a plurality of frequency curves and performing curve-fitting with the plurality of frequency curves to determine a best fit frequency.

At block 521, the interferometry application 139 could then be executed to determine a thickness of the tear film lipid layer and a thickness of the precorneal tear film based at least in part on the best fit frequency.

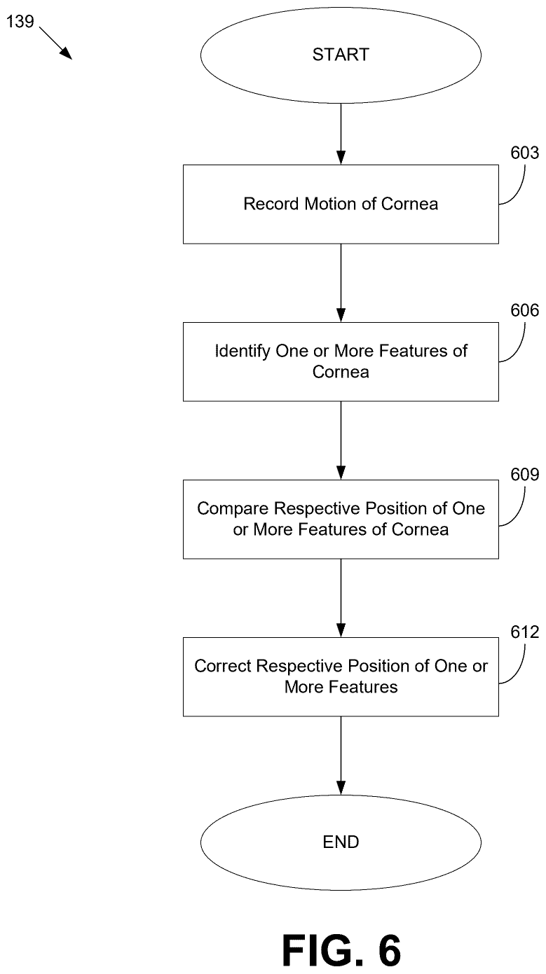

Referring now to FIG. 6, shown is a flowchart that provides one example of the operation of a portion of the interferometry application 139. The flowchart of FIG. 6 provides merely an example of the many different types of functional arrangements that can be employed to implement the operation of the depicted portion of the interferometry application 139. As an alternative, the flowchart of FIG. 6 can be viewed as depicting an example of elements of a method implemented within the interferometry system 100.

Beginning with block 603, the interferometry application 139 can be executed to record motion of the cornea during the point-by-point scan to produce a motion record. In some examples, the interferometry application 139 can be executed to record motion of the cornea using an area camera 119.

At block 606, the interferometry application 139 could then be executed to identify one or more features of the cornea from the motion record. In some examples, a lipid layer image can be used to identify one or more features.

At block 609, the interferometry application 139 could be executed to compare a respective position of the one or more features of the cornea in the motion record to the point-by-point scan. In some examples, the movement of the lipid layer images between two adjacent B-scans can be used to compare the respective position of the one or more features.

At block 612, the interferometry application 139 could be executed to correct the respective position of the one or more features in the point-by-point scan based at least in part on a detected change in the respective position between the motion record and the point-by-point scan. In some examples, a displacement calculation can be made from the center of the lipid layer images. The displacement calculation can then be used to correct the respective features in the interference signal. In some examples, a post processing algorithm can be incorporated into the interferometry application 139 similar to that used to correct motion artifacts in OCT retinal images.

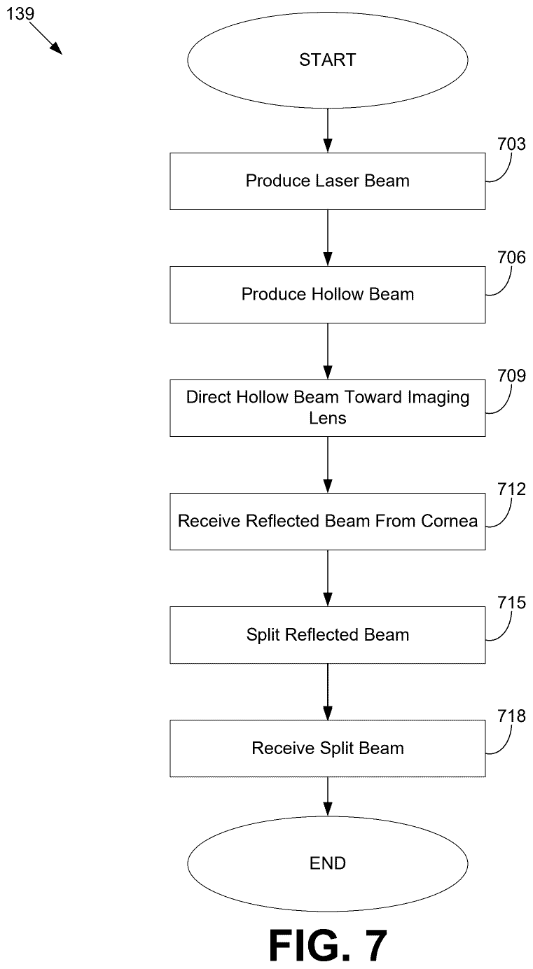

Referring now to FIG. 7, shown is a flowchart that provides one example of the operation of a portion of the interferometry application 139. The flowchart of FIG. 7 provides merely an example of the many different types of functional arrangements that can be employed to implement the operation of the depicted portion of the interferometry application 139. As an alternative, the flowchart of FIG. 7 can be viewed as depicting an example of elements of a method implemented within the interferometry system 100.

Beginning with block 703, the interferometry application 139 can be executed to produce a laser beam with a laser 106. In some examples, the laser beam can be a broadband laser beam (450 nm-750 nm).

At block 706, the interferometry application 139 could then be executed to produce a hollow beam from the laser beam using an axicon-pair 109. In some examples, the axicon-pair 109 can be configured to convert the laser beam from the laser 106 into a hollow beam. In some examples, the hollow beam can produce a Bessel spot with an extended depth of focus.

At block 709, the interferometry application 139 could be executed to direct the hollow beam toward an imaging lens 113 using a scanning mirror 103. In some examples, the imaging lens 113 can be an objective lens which has a curved focal plane similar to the surface of the cornea. In some examples, the imaging lens 113 can have a focal plane, the curvature of which is matched to that of a cornea surface, thereby producing a focal plan similar to the surface of the cornea. In some examples, the scanning mirrors 103 can be caused to rotate, directing the hollow beam through the imaging lens 113 to scan each point on the cornea at an angle perpendicular to the surface of the cornea.

At block 712, the interferometry application 139 could then be executed to receive a reflected beam from the cornea of the eye with a beam splitter 116b. In some examples, the beam splitter 116b can divert part of the reflected beam into an area camera 119. In some examples, the area camera 119 can use the part of the reflected beam to generate a motion record.

At block 715, the interferometry application 139 could then be executed to split the reflected beam with the beam splitter 116a to produce a split beam. In some examples, the interferometry application 139 could be executed to receive a reflected beam from the cornea of the eye with a beam splitter 116b and then be executed to split the reflected beam with the beam splitter 116a. In some examples, the beam splitter 116a can direct part of the reflected beam back through the scanning mirrors 103 and into a line scan camera 123.

At block 718, the interferometry application 139 could then be executed to receive the split beam with a line scan camera 123. In some examples, the line scan camera 123 can use the part of the reflected beam to generate an interference signal.

While embodiments of the present disclosure are described in connection with the Examples and the corresponding text and figures, there is no intent to limit the disclosure to the embodiments in these descriptions. On the contrary, the intent is to cover all alternatives, modifications, and equivalents included within the spirit and scope of embodiments of the present disclosure.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

An Interferometer for Tear Film Measurement with a Superior Sub-Micron Resolution

Various embodiments of the present disclosure measure the thickness of lipid layer (i.e., the outer most layer of the tear film) and overall tear film on the ocular surface of a human eye with a superior sub-micron resolution. The measured thickness can be used for dry eye diagnosis, evaluation and monitoring of clinical treatments for these conditions. Various embodiments of the present disclosure are based on the principle of interferometry: a broad light source in the visible region is utilized to induce the interferences between the different layers of the corneal surface (i.e., the lipid layer, and the aqueous layer of tear film, the epithelial layer of cornea), the detected signal of which can be processed and analyzed to retrieve the thickness of lipid layer and whole tear film with a specialized post-processing algorithm. Moreover, an objective with a curved focal plane is applied to cover a large area of the corneal surface (˜7 mm diameter), providing two dimensional “maps” of lipid layer and overall tear film. The advantages of these embodiments are to further provide a comprehensive and high-resolution evaluation of tear film by combing of a broadband light source, a specialized post-processing algorithm, and a customized objective.

A range of individual optical techniques based on different principles have been reported to noninvasively evaluate human tear film thickness. However, current evaluations of tear film thickness and related dynamics lack sufficient accuracy and sensitivity to detect discrete changes across the corneal surface, and fail to effectively guide ocular surface disease diagnosis and management. In fact, many methodologies to assess the tear film are iterative modifications of instruments developed several decades ago, including the tear film breakup time test, infrared thermography, and wavefront aberrometry. These methodologies do not take advantage of the advances of optics, photonics, and electronics with modern technology.

Various examples of the present disclosure leverage several innovations to construct a non-invasive, ultra-high-resolution optical imaging system that overcomes issues with current methods.

(i) Various embodiments use a broad bandwidth approach to achieve ultra-high resolution of the tear film measurements (e.g., ˜0.38 μm) that is not possible with other techniques, such as optical coherence tomography (OCT). The use of a broadband light source in an OCT system creates a dispersion mismatch between the reference and signal arms, which is difficult to resolve. The approach used herein avoids this mismatch with direct detection of the interference signal coming from the ocular surface, distinct from the reference arm or common path of the OCT. Second, the fast Fourier transform analysis approach used with OCT causes side-lobes with a non-Gaussian spectrum, reducing accuracy and sensitivity of OCT. The algorithm used herein isolates the tear film signal from the reflected signal and then processes it with the curve-fitting method, allowing for differentiation to improve resolution and accuracy.

(ii) Various embodiments herein have unprecedented accuracy for tear film lipid layer thickness determination. Prior methodologies estimate the tear film lipid layer thickness from the reflected intensity ratios among different wavelengths used in their light sources (usually red, green, and blue for most detectors). As the estimation is based only on the three-point wavelength (i.e., three-point dataset), its accuracy is limited. Additionally, the lipid layer is intimately interdigitated with the tear film but is proportionally much less substantial (e.g., 20-200 nm of the lipid layer compared to 3-5 μm of the tear film); thus, the reflected signal from the lipid layer inevitably intertwines with the signal from the tear film. A novel post-processing algorithm is developed to separate the signal of the lipid layer from that of the tear film, followed by a curve-fitting process. With this approach, the various embodiments of the present disclosure overcome the drawbacks of current methods and are able to determine the thickness of a lipid layer with unprecedented accuracy.

(iii) Various embodiments enable superior regional sensitivity on the ocular surface. With a laser source point-scanning system, regional sensitivity is determined by the spot size of light on the focal plane; thus, the regional sensitivity is also referred to as the spatial resolution in the x-y plane. To image the ocular surface in vivo, low magnification objectives combined with long depths of focus are usually used to mitigate eye motion artifacts. Such devices are limited by low regional sensitivity. In various embodiments described here, the focused light was reshaped from a Gaussian shape to a Bessel shape so that the depth of focus can be extended more than 10-fold. With this modification, these embodiments can use an objective with high magnification to greatly increase the spatial resolution (from ˜50-70 μm to ˜5-10 μm), which facilitates the exploration of associations between the lipid layer's microstructure and function.

(iv) Various embodiments image the corneal surface in a large field-of-view using a customized objective lens. Commercially available objective lenses can only measure the apical central region of the cornea due to their flat focal planes. One objective of the present disclosure is designed to match the scanned focal field with the spherical surface of the cornea. In summary, the present disclosure fills the gap of current techniques to reliably disentangle information about the tear film lipid layer and the overall tear film from the cornea surface.

Various embodiments of the present disclosure generally relate to the measurement of the tear film thickness on the ocular surface of the eye, with or without contact lenses, and more specifically, to the measurement of the thickness of both the outermost layer of the tear film (the lipid layer) and whole tear film. Such information could be used to diagnose ocular disease conditions such as dry eye disease or to evaluate the effectiveness of related ophthalmic treatments. Dry eye disease is one of the most frequent conditions for which patients seek medical eye care. Estimates from prevalence studies suggest that up to about one-third of the population suffers from dry eye disease, and these estimates are even greater in women and the elderly. Likewise, there are over 40 million contact lens wearers in the United States alone, all of whom have challenged tear films and ocular surface related to wearing contact lenses. Lastly, ocular allergy and infection are also of tremendous significance and importance, and this improved method to measure tear film dynamics would be of great utility in managing these conditions.

In various embodiments, an optical apparatus comprising: (i) a broadband (450 nm-750 nm) laser is collimated with a reflection collimator and enlarged with a pair of axicons to generate a tubular light beam, which produces a Bessel spot with extended depth of focus; (ii) scanning optics comprised of a 2D galvanometer scanner and a pair of biconvex lenses serving as a relay telescope; (iii) a customized imaging lens with a curved focal plane of the radius of 7.8 mm, which is mimic to the curvature of human eyes, focusing the scanned and relayed light from the scanning optics onto the corneal surface; (iv) one spectrometer to detect the signal reflected from the corneal surface. The spectrometer can comprise one collimating lens, one grating, one focusing lens and a high-speed line scan camera; and (v) one real-time auxiliary system for imaging the ocular surface to aid in aligning the system to the eye. The auxiliary system can comprise a light source, a liquid light guide with 5 mm in diameter, a collimating lens, and a video camera.

FIG. 1 shows the schematic layout of one example of the interferometric system. Red shapes describe the light path in the laser point-scanning system. The dotted line box on the right of the image indicates the auxiliary imaging system. The two light sources are separated with different polarization states; FIGS. 2A-D show a schematic motion correction with the aid of auxiliary system; FIG. 3 shows one example of the design of the customized objective, the requirements for which are 7 mm² field of view and collinearity of the lens chief rays to the 7.8 mm sphere radiuses. FIGS. 4A-C show the data processing from a precorneal tear film.

Various methods of measuring the thickness of the precorneal or pre-contact lens tear film, and specifically the lipid layer have been proposed. However, very few methods can integrate measures of the lipid layer and overall tear film on a single platform, fewer to measure the films with reliable accuracy and sufficient resolution, i.e., <1 μm. Compared to other methods, the current disclosure establishes a new algorithm to assess the dynamics of tear film and the lipid layer simultaneously at a superior resolution (˜0.38 μm) and a large field of view (˜7 mm).

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

Therefore, the following is claimed:

1. A system, comprising:
a computing device, comprising a processor and a memory; and
machine-readable instructions stored in the memory which, when executed by the processor, cause the computing device to at least:
obtain a point-by-point scan of a corneal surface of an eye from an interferometry system, the point-by-point scan including at least an interference signal;
obtain a large field-of-view of the corneal surface from an objective lens with a curved focal plane matched to a curvature of the corneal surface;
perform motion correction on the point-by-point scan;
filter noise from the interference signal;
separate a tear film lipid layer signal and a precorneal tear film signal from the filtered interference signal;
determine a best fit frequency for the tear film lipid layer signal and the precorneal tear film signal; and
determine a thickness of the tear film lipid layer and a thickness of the precorneal tear film based at least in part on the best fit frequency.

2. The system of claim 1, wherein the machine-readable instructions which, when executed, cause the computing device to perform motion correction further cause the computing device to at least:
record motion of the cornea during the point-by-point scan to produce a motion record;
identify one or more features of the cornea from the motion record;
compare a respective position of the one or more features of the cornea in the motion record to the point-by-point scan; and
correct the respective position of the one or more features in the point-by-point scan based at least in part on a detected change in the respective position between the motion record and the point-by-point scan.

3. The system of claim 1, wherein the interferometry system comprises:
a laser configured to produce a laser beam;
an axicon-pair configured to produce a hollow beam from the laser beam;
a scanning mirror to direct the hollow beam toward an imaging lens;
a beam splitter configured to receive a reflected beam from the cornea of the eye and split the reflected beam; and
a line scan camera to receive the split beam.

4. The system of claim 3, wherein the interferometry system further comprises an area camera configured to record motion of the cornea.

5. The system of claim 3, wherein the imaging lens comprises an objective lens having a focal plane with a curvature matched to that of the corneal surface.

6. The system of claim 1, wherein the machine-readable instructions which, when executed, cause the computing device to filter noise from the interference signal, further cause the computing device to at least subtract the interference signal from a standard interference signal.

7. A method, comprising:
obtaining, with an interferometry system, a point-by-point scan of a corneal surface of an eye, the point-by-point scan including at least an interference signal;
obtain a large field-of-view of a cornea of an eye from an objective lens with a curved focal plane matched to a curvature of a corneal surface;
performing motion correction on the point-by-point scan;
filtering noise from the interference signal;
separating a tear film lipid layer signal and a precorneal tear film signal from the filtered interference signal;
determining a best fit frequency for the tear film lipid layer signal and the precorneal tear film signal; and
determining a thickness of the tear film lipid layer and a thickness of the precorneal tear film based at least in part on the best fit frequency.

8. The method of claim 7, wherein performing motion correction on the point-by-point scan further comprises:
recording motion of the cornea during the point-by-point scan to produce a motion record;
identifying one or more features of the cornea from the motion record;
comparing a respective position of the one or more features of the cornea in the motion record to the point-by-point scan; and
correcting the respective position of the one or more features in the point-by-point scan based at least in part on a detected change in the respective position between the motion record and the point-by-point scan.

9. The method of claim 8, wherein recording motion of the cornea uses an area camera.

10. The method of claim 7, wherein filtering noise from the interference signal further comprises at least subtracting the interference signal from a standard interference signal.

11. The method of claim 7, wherein obtaining, with the interferometry system, the point-by-point scan comprises:
producing a laser beam with a laser;
producing a hollow beam from the laser beam using an axicon-pair;
directing the hollow beam toward an imaging lens using a scanning mirror;
receiving a reflected beam from the cornea of the eye with a beam splitter;
splitting the reflected beam with the beam splitter to produce a split beam; and
receiving the split beam with a line scan camera.

12. The method of claim 11, wherein the imaging lens has a focal plane with a curvature matched to that of the corneal surface, the imaging lens producing a focal length similar to that of the cornea.

13. A system, comprising:
an interferometry system;
a computing device, comprising a processor and a memory; and
machine-readable instructions stored in the memory which, when executed by the processor, cause the computing device to at least:
obtain an interference signal from a corneal surface of an eye using the interferometry system;
obtain a large field-of-view of the corneal surface from an objective lens with a curved focal plane matched to a curvature of the corneal surface;
filter noise from the interference signal to produce a deduction signal;
separate a tear film lipid layer signal and a precorneal tear film signal from the deduction signal;
determine a respective best fit frequency for both of the tear film lipid layer signal and the precorneal tear film signal; and
determine a thickness of the tear film lipid layer and a thickness of the precorneal tear film based at least in part on the respective best fit frequencies.

14. The system of claim 13, wherein the machine-readable instructions, when executed, further cause the computing device to at least:
using an area camera of the interferometry system, record motion of the cornea to generate a motion record; and
perform motion correction on the interference signal based at least in part on the motion record.

15. The system of claim 14, wherein the machine-readable instructions which, when executed, cause the computing device to perform motion correction, further cause the computing device to at least:
identify one or more features of the cornea from the motion record;
compare a respective position of the one or more features of the cornea in the motion record to the interference signal; and
correct the respective position of the one or more features in the interference signal based at least in part on a detected change in the respective position between the motion record and the interference signal.

16. The system of claim 13, wherein the machine-readable instructions which, when executed, cause the computing device to obtain an interference signal from a cornea of an eye using the interferometry system, further cause the computing device to at least:
direct a hollow laser beam of the interferometry system toward the cornea through an imaging lens; and
receive a reflected beam from the cornea of the eye to obtain the interference signal.

17. The system of claim 16, wherein the imaging lens comprises an objective lens having a focal plane with a curvature matched to that of the corneal surface.

18. The system of claim 13, wherein the machine-readable instructions which, when executed, cause the computing device to filter noise from the interference signal, further cause the computing device to at least subtract the interference signal from a standard interference signal.

19. The system of claim 13, wherein the machine-readable instructions which, when executed, cause the computing device to determine a best fit frequency for both of the tear film lipid layer signal and the precorneal tear film signal, further cause the computing device to at least:
simulate a plurality of frequency curves; and
perform curve-fitting with the plurality of frequency curves to determine a best fit frequency.

20. The system of claim 13, wherein the interferometry system comprises:
a laser configured to produce a laser beam;
an axicon-pair configured to produce a hollow beam from the laser beam;
a scanning mirror to direct the hollow beam toward an imaging lens;
a beam splitter configured to receive a reflected beam from the cornea of the eye and split the reflected beam; and
a line scan camera to receive the split beam.
